(19) United States
(12) Patent Application Publication
(10) Pub. No.: US 2008/0044851 A1
(43) Pub. Date: Feb. 21, 2008

(54) Compositions and methods for removal of DNA from a sample

(75) Inventors: Jerome Jendrisak, Madison, WI (US); Haiying L. Grunenwald, Belville, WI (US)

Correspondence Address: MEDLEN & CARROLL, LLP, 101 HOWARD STREET, SUITE 350, SAN FRANCISCO, CA, 94105, US

(73) Assignee: Epicentre Technologies, Madison, WI (US)

(21) Appl. No.: 11/810,309

(22) Filed: Jun. 4, 2007

Related U.S. Application Data
(60) Provisional application No. 60/810,421, filed on Jun. 2, 2006.

(51) Int. Cl. C12N 9/16; C12P 1/00

(52) U.S. Cl. 435/41; 435/196

(57) ABSTRACT

The present invention provides compositions and methods for digesting DNA. In particular, the present invention provides enzymes mixtures that provide enhanced DNA digestion and methods of using the enzyme mixtures to eliminate or reduce undesired DNA molecules from a sample of interest.

The present application claims priority to U.S. Provisional Application Ser. No. 60/810,421, filed Jun. 2, 2006, the disclosure of which is herein incorporated by reference in its entirety.

### FIELD OF THE INVENTION

The present invention provides compositions and methods for digesting DNA. In particular, the present invention provides enzyme mixtures that provide enhanced DNA digestion and methods of using the enzyme mixtures to eliminate or reduce undesired DNA molecules from a sample of interest.

### BACKGROUND

A wide range of life sciences procedures benefit from the removal of DNA from samples. For example, research, diagnostic, and therapeutic methods that require the isolation and/or amplification of RNA from a sample of interest often require that DNA from the sample be removed or substantially eliminated. DNA removal is needed for reverse transcription polymerase chain reactions (RT-PCR), where even trace amounts of contaminating DNA can cause undesired false positive results. RNA isolated from some tissues, such as spleen, kidney, or thymus, and RNA isolated from transfected cells tends to contain high levels of DNA contamination, resulting in particular need for contaminant removal. Additionally, certain disease conditions are associated with DNA accumulation. Reduction of the DNA reduces symptoms associated with the diseases.

A variety of techniques have been developed to help minimize the problem of contaminating or undesired DNA. Time-consuming and labor-intensive methods such as cesium chloride centrifugation are effective, but often not practical. Extraction methods, precipitation methods, and filter-based absorptive technologies have been developed (discussed in U.S. Pat. Pub. No. 20050233333, herein incorporated by reference in its entirety; see also U.S. Pat. Pub. No. 20050026159, herein incorporated by reference in its entirety), but are often expensive and labor-intensive, cause undesired loss of RNA, and/or are not sufficiently effective at removing DNA from RNA.

A common method for removal of DNA from a sample comprises contacting the sample with a type I deoxyribonuclease (DNase). DNases are phosphodiesterases capable of hydrolyzing polydeoxyribonucleic acid molecules. DNases are classified as type I or type II. A “type I DNase”, as used herein, is an endodeoxyribonuclease that digests single-stranded and double-stranded DNA to short oligonucleotides having a 5′-phosphate and a 3′-hydroxyl group. A type I DNase is exemplified by human, bovine and porcine pancreatic DNase I (human pancreatic DNase I is described in U.S. Pat. Nos. 6,569,660; 5,830,744; 6,391,607; 6,348,343; and Pan et al., Protein Science, 7: 628, 1998; bovine pancreatic DNase I is described in Worrall and Connolly, J. Biol. Chem., 265: 21889, 1990, and in U.S. Pat. Publ. No. 2004/0219529; porcine pancreatic DNase I is described in Mori et al., Biochim. Biophys. Acta 1547: 275, 2001; herein incorporated by reference in their entireties). A type II DNase produces nucleotides having a 3′-phosphate on hydrolysis of DNA.

While DNases are primarily recognized as digestive enzymes, they have been shown to play a role in a variety of biological processes, including genetic recombination, repair of DNA damage, restriction of foreign DNA, and transport of DNA into cells. DNases from a variety of species have been identified and purified. Porcine and bovine type I DNases have been known for decades and are well characterized. Human type I DNases have also been identified, cloned, expressed, and engineered (see e.g., U.S. Pat. Nos. 6,569,660; 5,830,744; 6,391,607; 6,348,343; and Pan et al., Protein Science, 7: 628, 1998, herein incorporated by reference in their entireties).

While type I DNases have many favorable features, existing type I DNases and methods of using them do not eliminate sufficient amounts of DNA as are needed for methods that are sensitive to trace amounts of contaminating DNA, such as RT-PCR or for preparing RNA samples (e.g., capped and polyadenylated RNA) for gene expression analysis, or for preparing RNA for in vivo therapeutic applications in humans or animals (e.g., to transform cells and express proteins in cells).

What is needed are improved compositions and methods for eliminating or reducing more of the undesired DNA in a sample than is possible with presently available compositions of type I DNase, including DNase I, and presently available methods. What is needed are improved compositions and methods for removing undesired DNA from a sample that are time-efficient and inexpensive.

### DESCRIPTION OF THE DRAWINGS

FIG. 1 shows the results of Example 4, and in particular an agarose gel analysis of DNA remaining after DNase I digestion using commercially available DNase I enzymes, as well as the combination of DNase I and Exonuclease I.

FIG. 2 shows the results of Example 5, and in particular an agarose gel analysis of DNA remaining after DNase I digestion using commercially available DNase I enzymes, as well as the combination of DNase I and Exonuclease I.

### SUMMARY OF THE INVENTION

The present invention provides compositions and methods for digesting DNA. In particular, the present invention provides enzyme mixtures that provide enhanced DNA digestion and methods of using the enzyme mixtures to eliminate or reduce undesired DNA molecules from a sample of interest.

For example, the present invention provides a composition comprising a mixture of a conventional type I DNase used for DNA digestion and a second complementary DNase, wherein the complementary DNase acts synergistically to enhance the function of the type I DNase. When a type I DNase, such as bovine, porcine, or human DNase I, is used to digest double-stranded DNA, which is commonly present in biological samples, the activity of the enzyme appears to decrease over time as the reaction proceeds. An understanding of the mechanism is not necessary to practice the present invention and the present invention is not limited to any particular mechanism of action. However, it is contemplated that the end-products of the type I DNase degradation of DNA result in end-product inhibition of the type I DNase, including DNase I. For example, Vanecko and Laskowski (J. Biol. Chem., 236: 3312, 1961) showed that autoretardation of enzyme activity is a result of the continuous formation of products that are poorer substrates than those from which they are derived, thus acting as inhibitors against the digestion of larger DNA fragments. That is, the small oligo products of digestion by the type I DNase act as end-product inhibitors of the enzyme, thus limiting the reaction. The second complementary DNase enzyme provided in the compositions, kits and methods of the present invention enzymatically degrade the single-stranded small oligo products of the end-product-inhibited type I DNase enzyme, thereby preventing the inhibition and enhancing DNA digestion by the type I DNase. Removing the small end-product oligo inhibitors from the reaction also results in better and more complete digestion of larger DNA that is otherwise not digested due to inhibition of the type I DNase. The larger DNA that is not removed is a source of background for RT-PCR, and can result in low purity RNA for other applications.

Thus, the present invention pertains to compositions, kits and methods for improving digestion of DNA using a type I DNase, including, without limitation, bovine, porcine, or human DNase I. The second complementary DNase enzyme of the present invention comprises a single-strand-specific 3′-to-5′ exodeoxyribonuclease that lacks ribonuclease activity, but that digests oligodeoxyribonucleotides having a free 3′-hydroxyl group to 5′-monodeoxyribonucleotides. Exonuclease I is an exemplary complementary DNase enzyme for use in a composition, kit or method of the present invention. One example of an exonuclease I that can be used as the second complementary enzyme is exonuclease I from Escherichia coli. Other organisms that contain exonuclease I enzyme and homologues can be determined by the skilled artisan through BLAST homology searches of data available in data bases, e.g. GenBank, the TIGR database, and other data bases. At the time of writing those other organisms that contain exonuclease I enzyme and homologues included Actinobacillus succinogenes, Actinobacillus pleuropneumoniae, Alteromonas macleodii, Azotobacter vinelandii, Bacteriophage KVP40, Bradyrhizobium japonicum, Buchnera aphidicola, Buchnera sp. APS, Candidatus Blochmannia floridanu, Candidatus Blochmannia pennsylvanicus, Candidatus Pelagibacter ubique, Chromobacterium violaceum, Chromohalobacter salexigens, Colwellia psychrerythraea, Erwinia carotovora, Francisella tularensis, gamma proteobacterium KT 71, Haemophilus ducreyi, Haemophilus influenzae, Haemophilus somnus, Hahella chejuensis, Idiomarina loihiensis, Idiomarina baltica, Mannheimia succiniciproducens, Marinobacter aquaeolei, Marinomonas sp., Methylococcus capsulatus, Microbulbifer degradans, Mycoplasma mycoides, Nitrosomonas europaea, Nitrosomonas eutropha, Oceanospirillum sp., Pasteurella multocida, Photobacterium profundum, Photobacterium sp., Photorhabdus luminescens, Polaromonas naphthalenivorans, Polaromonas sp., Pseudoalteromonas atlantica, Pseudoalteromonas haloplanktis, Pseudoalteromonas tunicate, Pseudomonas aeruginosa, Pseudomonas fluorescens, Pseudomonas putida, Pseudomonas syringae, Ralstonia solanacearum, Reinekea sp., Rhodoferax ferrireducens, Rhodopseudomonas palustris, Roseobacter sp., Rubrivivax gelatinosus, Salmonella enterica, Salmonella typhimurium, Shewanella amazonensis, Shewanella baltica, Shewanella denitrificans, Shewanella frigidimarina, Shewanella oneidensis, Shewanella putrefaciens, Shewanella sp., Shigella boydii, Shigella dysenteriae, Shigella flexneri, Shigella sonnei, Sodalis glossinidius, Vibrio cholerae, Vibriofischeri, Vibrio parahaemolyticus, Vibrio sp., Vibrio splendidus, Vibrio vulnificus, Xanthomonas axonopodis, Xanthomonas campestris, Xanthomonas oryzae, Xylella fastidiosa, Yersinia bercovieri, Yersinia frederiksenii, Yersinia intermedia, Yersinia mollaretii, Yersinia pestis, and Yersinia pseudotuberculosis. However, complementary DNases having the desired properties, from any organism now known or later discovered find use with the present invention.

The present invention comprises a method for removing undesired DNA in a sample, the method comprising contacting the sample with a mixture of a type I DNase and a complementary DNase enzyme under conditions wherein the undesired DNA is digested. The method comprises digestion of the undesired DNA using a mixture of both the type I DNase and the complementary DNase enzyme, whereby the undesired DNA in the sample is digested. In some embodiments, the type I DNase and the complementary DNase are provided as a composition comprising a mixture of both enzymes. In some embodiments, the type I DNase and the complementary DNase enzyme are provided as separate compositions in a kit that are combined upon addition to the sample containing the undesired DNA. In such embodiments, the method further comprises the steps of adding the composition of the type I DNase and the complementary DNase to the sample, mixing the sample containing the type I DNase and the complementary DNase, and incubating the sample under reaction conditions, whereby the undesired DNA in the sample is digested. In some embodiments, both enzymes are added together to a sample. In preferred embodiments, a mixture of the two enzymes is added to the sample. The present invention is not limited by the amount of or ratio of the two enzymes. Whether provided together as a single composition or as individual enzymes in a kit, in some embodiments, the compositions comprising the mixture or the individual enzymes in the kit are provided RNase-free or substantially RNase-free. Likewise, in some embodiments, the enzymes are provided free of or substantially free of proteases. The type I DNase and the complementary DNase enzyme may be provided in pure form (e.g., chromatographically pure, filter purified, etc.) or may be provided in a partially purified or isolated form.

In some preferred embodiments, the enzymes are in solution (e.g., in a buffer). In some embodiments, the enzymes are provided, either as a single mixed composition together or as individual components in a kit, e.g., in lyophilized form.

In some embodiments, nucleic acid sequences (e.g., expression vectors) that encode the enzymes of the present invention are provided (e.g., in one or more host cells, transgenically for in vivo expression, etc.). In some embodiments, a recombinant construct that expresses both enzymes is used. In some such embodiments, the enzymes are expressed as a conjugate, wherein a protein complex contains both enzymes (e.g., attached by a linker or fused together). In some embodiments, one or both of the nucleic acid sequences (e.g., for the type I DNase and the complementary DNase enzyme) are inserted into and expressed from the DNA of the host cell (e.g., into the host's genomic DNA). In some embodiments, one or both of the nucleic acid sequences (e.g., for the type I DNase and the complementary DNase enzyme) are inserted into the DNA of the host cell (e.g., into the host's genomic DNA) using a transposome (e.g., made by first cloning each said nucleic acid sequence that is joined to a suitable RNA polymerase promoter sequence into a pMod™ vector (Epicentre Biotechnologies, Madison, Wis., USA) to make an artificial EZ-Tn5™ transposon, which is then incubate in vitro with EZ-Tn5™ transposase (Epicentre), to obtain an EZ-Tn5™ transposome, which is then used to transform the host cell and select for transpositions and expression of the enzyme activity, according to instructions of the supplier (Epicentre) and as known in the art.

The present invention is not limited by the nature of the sample that is treated with the enzymes of the present invention. In some embodiments, the sample is provided in vivo, ex vivo, in culture, or in vitro. In some embodiments, the sample is a cell extract or lysate. In some embodiments, the sample is a preparation from a first DNA removal method, where the enzymes of the present invention are used to further eliminate DNA from the sample.

The present invention also provides kits for use with or in the compositions and methods of the present invention. In some embodiments, the kits comprise a type I DNase and a complementary DNase. In some embodiments, the enzymes are provided in concentrated form (e.g., 5×, 10×, etc.) such that they are diluted for use. The kits may also contain one or more additional components that find use in the compositions and methods of the present invention including, but not limited to, containers for housing reagents, buffers, control reagents (e.g., control RNA or DNA, etc.), RT-PCR reagents (e.g., polymerases, primers, reverse transcriptases, labels, probes, etc.), real-time RT-PCR reagents, such as Taqman reagents (U.S. Pat. No. 5,210,015, herein incorporated by reference in its entirety), in vitro transcription reagents (e.g., RNA polymerases), cell culture reagents (e.g., culture media, transfection reagents), cloning reagents (e.g., restriction enzymes, host cells), DNase removal agents/inactivators (e.g., EGTA, proteinaceous inhibitors, proteases, DNase Removal Reagent from Ambion, Austin, Tex., USA), RNA purification reagents or components (e.g., filters, columns, solvents, resins, etc.), instructions for use (e.g., instructions required by the FDA for diagnostic or therapeutic products), software, therapeutic agents (e.g., delivery devices, compounds to be co-administered, etc.), and the like.

Thus, in some embodiments, the present invention provides a composition (e.g., kit, reaction mixture, container, etc.) comprising a purified type I DNase enzyme, such as human, bovine, porcine or another homologous DNase I and a purified complementary 3′-to-5′ exodeoxyribonuclease enzyme, such as exonuclease I. In some embodiments, the enzymes are provided together in a buffer (e.g., comprising calcium and a divalent ion such as magnesium or manganese; comprising a surfactant, e.g., Triton X100 or equivalent). In some embodiments, the composition comprises a sample containing RNA molecules. In some embodiments, the composition comprises a DNase enzyme component that consists of a purified type I DNase enzyme and a purified complementary 3′-to-5′ exodeoxyribonuclease enzyme (i.e., the composition may contain other components, including other enzymes, but the only DNase enzymes present are a purified type I DNase enzyme and a purified complementary 3′-to-5′ exodeoxyribonuclease enzyme). In some embodiments, the composition comprises an enzyme component that consists of a purified type I DNase enzyme and a purified complementary 3′-to-5′ exodeoxyribonuclease enzyme (i.e., the composition may contain other components, but the only enzymes present are a purified type I DNase enzyme and a purified complementary 3′-to-5′ exodeoxyribonuclease enzyme). In some embodiments, the composition consists of a purified type I DNase enzyme and a purified complementary 3′-to-5′ exodeoxyribonuclease enzyme in a suitable storage buffer.

The compositions and kits of the present invention may further comprise one or more additional enzymes or proteins, including, but not limited to, DNA polymerases (e.g., E. coli DNA polymerase, Taq polymerase, Tth polymerase, Pfu polymerase, KOD-1 polymerase, Pwo polymerase, Tfl polymerase, Psp polymerase, Tli polymerase, and variants thereof), ribonuclease (e.g., RNases, RNaseH), RNA polymerases (e.g. T7-type RNA polymerases such as T7 RNA polymerase, T3 RNA polymerase, and SP6 RNA polymerase; miniV RNA polymerase (EPICENTRE); E. Coli RNA polymerase), reverse transcriptases (AMV, MMLV, HIV-1, RNase H-MMLV, Improm-II (Promega), Omniscript (Qiagen), ThermoScript RNase H⁻ (Invitrogen), and RNase H⁻ SuperScript I, II, or III (Invitrogen), proteases, or lipases.

In some embodiments, the present invention provides a method of digesting DNA in a sample, comprising: treating a sample comprising DNA with an enzyme mixture comprising a type I DNase enzyme and a complementary 3′-to-5′ single-strand-specific exodeoxyribonuclease, such as exonuclease I. In some embodiments, the present invention provides a method for DNA removal comprising treating a sample suspected of comprising DNA with an enzyme mixture comprising a type I DNase enzyme, such as DNase I, and a complementary 3′-to-5′ single-strand-specific exodeoxyribonuclease, such as exonuclease I enzyme. The method may involve one or more of: degradation of contaminating DNA after RNA isolation, clean-up of RNA prior to reverse transcription (e.g. for RT-PCR or other amplification methods), clean-up of RNA after in vitro transcription, removal of DNA from protein samples, prevention of clumping of cultured cells, creating a fragmented library of DNA sequences (e.g., for in vitro recombination reactions, microarray reactions, etc.), on-column DNA removal before elution of RNA from a solid support, RNA polymerase synthesis of RNA probes, removing membrane-bound DNA fragments from cells (e.g., cultured cells), and removing or reducing DNA in mucus in vivo for therapeutic or research uses.

For example, the present invention provides a method for reverse transcription comprising: incubating a sample suspected of comprising DNA with an enzyme mixture of the present invention prior to incubation with a reverse transcriptase enzyme (e.g., under conditions such that RNA in the sample is reverse transcribed). Additionally, for example, the present invention provides a method for making a cDNA comprising: incubating a sample suspected of comprising DNA with an enzyme mixture of the present invention, removing or inactivating the enzyme mixture, then incubating the sample with a reverse transcriptase enzyme, and a primer under conditions such that cDNA is made from an RNA molecule in the sample.

The enzyme mixtures of the present invention may be combined in kits with optimized and quantity-matched reagents for the above methods (e.g., RT-PCR reagents, in vitro transcription reagents, etc.) such that the methods can be conducted in the least number of steps possible.

Definitions

To facilitate understanding of the invention, a number of terms are defined below.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and cell lysates. Biological samples include urine and blood products, such as plasma, serum and the like. Such examples are not however to be construed as limiting the sample types applicable to the present invention.

As used herein, the term “subject” refers to any animal (e.g., a mammal), including, but not limited to, humans, non-human primates, rodents, and the like, which is to be the recipient of a particular diagnostic test or treatment. Typically, the terms “subject” and “patient” are used interchangeably herein in reference to a human subject.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

As used herein, the term “in vitro” refers to an artificial environment and to processes or reactions that occur within an artificial environment. In vitro environments are exemplified by, but are not limited to, test tubes and cell cultures. The term “in vivo” refers to the natural environment (e.g., an animal or a cell) and to processes or reactions that occur within a natural environment.

As used herein, the terms “isolated,” “to isolate,” “isolation,” “purified,” “to purify,” “purification,” and grammatical equivalents thereof as used herein, unless specified otherwise, refer to the reduction in the amount of at least one contaminant (such as protein and/or nucleic acid sequence) from a sample. Thus purification results in an “enrichment,” i.e., an increase in the amount of a desirable protein and/or nucleic acid sequence in the sample.

A “type I DNase”, as used herein, is an endodeoxyribonuclease that digests single-stranded and double-stranded DNA to short oligonucleotides having a 5′-phosphate and a 3′-hydroxyl group. A type I DNase is exemplified by human, bovine and porcine pancreatic DNase I (human pancreatic DNase I is described in U.S. Pat. Nos. 6,569,660; 5,830,744; 6,391,607; 6,348,343; and Pan et al., Protein Science, 7: 628, 1998; bovine pancreatic DNase I is described in Worrall and Connolly, J. Biol. Chem., 265: 21889, 1990; porcine pancreatic DNase I is described in Mori et al., Biochim. Biophys. Acta 1547: 275, 2001; herein incorporated by reference in their entireties).

A “complementary DNase” or a “complementary DNase enzyme”, as used herein, means a single-strand-specific 3′-to-5′ exodeoxyribonuclease that lacks ribonuclease activity, but that digests oligodeoxyribonucleotides having a free 3′-hydroxyl group to monodeoxyribonucleotides having a 5′-phosphate. Exonuclease I is an exemplary complementary DNase.

The term “variant” of a protein (such as a type I DNase or a complementary DNase) as used herein is defined as an amino acid sequence that differs by insertion, deletion, and/or substitution (e.g., conservative substitution) of one or more amino acids from the protein of which it is a variant. The term “conservative substitution” of an amino acid refers to the replacement of that amino acid with another amino acid that has a similar hydrophobicity, polarity, and/or structure. For example, the following aliphatic amino acids with neutral side chains may be conservatively substituted one for the other: glycine, alanine, valine, leucine, isoleucine, serine, and threonine. Aromatic amino acids with neutral side chains which may be conservatively substituted one for the other include phenylalanine, tyrosine, and tryptophan. Cysteine and methionine are sulphur-containing amino acids which may be conservatively substituted one for the other. Also, asparagine may be conservatively substituted for glutamine, and vice versa, since both amino acids are amides of dicarboxylic amino acids. In addition, aspartic acid (aspartate) may be conservatively substituted for glutamic acid (glutamate) as both are acidic, charged (hydrophilic) amino acids. Also, lysine, arginine, and histidine may be conservatively substituted one for the other since each is a basic, charged (hydrophilic) amino acid. Guidance in determining which and how many amino acid residues may be substituted, inserted or deleted without adversely affecting biological and/or immunological activity may be found using computer programs well known in the art, for example, DNASTAR software. In one embodiment, the sequence of the variant has at least 95% identity, at least 90% identity, at least 95% identity, at least 80% identity, at least 75% identity, at least 70% identity, and/or at least 65% identity with the sequence of the protein in its wild-type or most predominant wild-type form.

### DETAILED DESCRIPTION OF THE INVENTION

The present invention provides compositions, kits and methods for improved DNA digestion by combining a second complementary DNase enzyme with a type I DNase. Experiments conducted during the development of the present invention demonstrated that a second complementary DNase enzyme, capable of digesting the cleavage products of type I DNase, synergistically improved DNA digestion. While an understanding of the mechanism of action is not necessary to practice the present invention, it is contemplated that the second complementary DNase enzyme, which is single-strand-specific and therefore cannot digest double-stranded DNA itself, functions primarily by digesting the single-stranded cleavage products of the type I DNase enzyme, thereby preventing and/or removing end-product inhibition of the type I DNase and permitting the type I DNase to maintain high activity.

Thus, the present invention provides compositions and methods using a type I DNase and a second complementary DNase enzyme for a wide variety of applications where DNA digestion is desired. Certain specific embodiments of the present invention are described below to illustrate various aspects of the invention. The present invention is not limited to these embodiments.

Preferred enzymes of the present invention that may be used as the second complementary DNase enzyme are those that have the following properties: 1) capable of exodeoxyribonuclease digestion of DNA products generated by the type I DNase (e.g., digests single-stranded deoxyribonucleic acid, but not ribonucleic acid, having a 3′-hydroxyl terminus); 2) possesses DNase activity under reaction conditions under which the type I DNase has activity (e.g., functions in the same buffer, at the same temperature, etc.); 3) does not interfere with the type I DNase activity; 4) the combined deoxyribonuclease activity, when used with the type I DNase, is synergistic rather than additive (i.e., the amount of DNA digestion is greater than the sum of each enzyme acting alone); and 5) the complementary DNase degrades single-stranded DNA to single nucleotides. Enzymes that fit the above criteria can be identified by the use of straightforward screening methods. For example, one or more of the following test protocols may be used to identify or characterize enzymes for use in the present invention: 1) exposure to different DNA substrates (e.g., single-stranded, double-stranded, circular, 3′-hydroxyl termini, 5′-hydroxyl termini, etc.) and determination of products generated; 2) treatment of double-stranded DNA substrates in the presence or absence of a type I DNase and/or in the presence or absence of a potential complementary DNase to determine additive or synergistic benefits; 3) assay for DNase activity to determine any inhibition of the type I DNase; and the like. The Examples section, below, provides exemplary methods that may be used.

In some preferred embodiments, the second complementary enzyme is exonuclease I enzyme, such as exonuclease I encoded by E. coli. Exonuclease I catalyzes the removal of nucleotides from single-stranded DNA in the 3′ to 5′ direction (see, Lehman and Nussbaum, J. Biol. Chem., 239:2628 (1964); Kushner et al., Proc. Natl. Acad. Sci. USA 68:824 (1971); and Kushner et al., Proc. Natl. Acad. Sci. USA 69:1366 (1972), herein incorporated by reference in their entireties). Exonuclease I does not digest double-stranded DNA (dsDNA). Although exonuclease I prefers the presence of magnesium and a free 3′-hydroxyl terminus for activity, it is active under a wide variety of buffer conditions and can be added directly into most reaction mixes. Exonuclease I can be heat-inactivated by incubation at 80° C. for 15 minutes. Exonuclease I may be used as commercially available from a variety of sources (e.g., EPICENTRE® Biotechnologies, Madison, Wis.; New England Biolabs, Ipswich, Mass.; Molecular Cloning Laboratories, South San Francisco, Calif.; USB Corp., Cleveland, Ohio; Yorkshire Bioscience Ltd., York, United Kingdom). Variant exonuclease I enzymes may be used, so long as they retain nuclease activity. Variants include, but are not limited to, fragments, chimeras (e.g., containing an affinity label to assist with purification), and deletions, insertions, and substitution mutants (e.g., to improve purification, stability, and the like). The exonuclease may be recombinantly produced or may be biochemically purified from a host organism. It is also contemplated that chimeras containing active functional domains from a type I DNase and a second complementary enzyme are used.

In some embodiments, the complementary DNase enzyme can be a mammalian equivalent of exonuclease I (e.g., human exonuclease, including the human enzyme referred to in the literature as TREX1/DNase III (Höss et al., EMBO J. 18: 3868, 1999; Morita et al., Mol Cell Biol, 24: 6719, 2004). Such enzymes are contemplated to be particularly useful in the human in vivo methods of the present invention.

Common type I DNases that find use in the compositions and methods of the invention include mammalian DNase I including, but not limited to, bovine, porcine, and human DNase I. Bovine pancreatic DNase I is an endonuclease that cleaves both single-stranded DNA (ss-DNA) and double-stranded DNA (ds-DNA) to produce primarily 5′-P-dinucleotides and 5′-P-oligonucleotides (see Enzymology Primer for Recombinant DNA Technology, Academic Press, Eun, Chapter 3, Nucleases, herein incorporated by reference in its entirety). DNase I degrades dsDNA in a sequence-nonspecific manner. The enzyme requires divalent cations (e.g., Ca²⁺ and Mg²⁺ or Mn²⁺) as cofactors for full double-strand cutting activity. In the presence of Mg²⁺ alone as a cofactor, DNase I exhibits nicking activity and the single-stranded nucleolytic activity is more discriminatory. Bovine pancreatic DNase I is a mixture of four glycoprotein components of similar catalytic activity: DNase A (major), B, C, and D. For most purposes, a mixture of the four is a suitable catalyst. DNase I is distinct from DNase II, a lysosomal DNase found in various organs such as the thymus, liver, and spleen. DNase I prefers a duplex region for cleavage, wherease DNase II prefers a single-stranded region for its activity. Furthermore, DNase II differs from DNase I in its optimal pH and the requirement for Mg²⁺. DNase II generates 3′-phosphorylated oligonucleotides as the predominant products. Optimal reaction conditions and kinetic parameters for DNase I are well characterized (see Enzymology Primer for Recombinant DNA Technology, supra). Although ssDNAs are substrates for DNase I, dsDNAs are 100 to 500 times better substrates (Drew, J., Mol. Biol. 176: 535, 1984).

In some embodiments of the present invention, the type I DNase enzymes and complementary DNase enzymes can be obtained from commercial vendors (e.g., bovine pancreas DNase I, Epicentre Biotechnologies®, Madison, Wis.; Worthington Biochemical Corporation, Lakewood, N.J.; Sigma-Aldrich; New England Biolabs; Ambion Inc., Austin, Tex.; Promega Corporation, Madison, Wis.). The DNase enzymes can be purified from cells or tissue or can be prepared recombinantly (e.g., grown in Piciapastoris, E. coli, etc.; see Worrall and Connolly, J. Biol. Chem. 265: 21889, 1990). Porcine type I DNase is described in Mori et al., Biochim. Biophys. Acta 1547: 275, 2001). Native and variant human DNases are described in U.S. Pat. Nos. 6,569,660, 5,830,744, 6,391,607, 6,348,343, and Pan et al., Protein Science 7: 628, 1998), herein incorporated by reference in their entireties.

The enzyme mixtures of the present invention find use in any method where it is desirable or required that digestion of DNA, at least a portion of which is double-stranded DNA, is essentially complete. For example, the enzyme mixtures of the present invention may be used in a wide variety of molecular biology applications where essentially complete removal of DNA is desired. Such methods include, but are not limited to: degradation of contaminating DNA after RNA isolation; removal of DNA from RNA prior to RT-PCR; to remove DNA templates, including any associated vectors, from RNA after in vitro transcription or in vitro RNA amplification reactions; to remove genomic and other cellular DNA from RNA prior to synthesis of cDNA by reverse transcription (e.g., prior to PCR or prior to RNA amplification by attaching an RNA polymerase promoter to the cDNA and then transcribing the cDNA using an RNA polymerase that recognizes the promoter (e.g. Van Gelder, R N. et al. 1990, Proc. Natl. Acad. Sci. USA 87, 1663; U.S. Patent Application No. 2005/0153333 by Sooknanan) removal of DNA from protein samples, especially from therapeutic or diagnostic proteins; prevention of clumping when handling cultured cells, especially cells for therapeutic use; RNA polymerase synthesis of RNA probes; removing membrane bound DNA fragments from cells (cultured cells), especially for therapeutic or clinical research use; or removing DNA from RNA for in vivo therapeutic use in humans or animals or for clinical research. In some embodiments, the enzyme mixtures of the present invention find use for removing DNA from RNA that is synthesized using an in vitro transcription or RNA amplification reaction that synthesizes sense RNA (e.g., using the method described in U.S. Patent Application No. 2005/0153333 or other methods known in the art), which RNA is capped and polyadenylated using any method known in the art, and used for transforming human or animal cells. In some embodiments, the human or animal cells that are transformed using the capped and polyadenylated RNA that is treated with the enzyme mixture of the present invention are antigen-presenting cells (e.g., antigen-presenting cells selected from among dendritic cells, macrophage cells, epithelial cells, or artificial antigen-presenting cells, whether obtained from a patient or made in culture using methods known in the art). In some embodiments, the RNA that is treated with the enzyme mixture of the present invention is translated into a polypeptide in vivo in a cell, either in culture or in a cell in an organism (e.g., in a human or animal organism).

Indeed, the enzyme mixtures of the present invention may be used to improve any sample preparation method that is characterized by DNA contamination. Improvements include, but are not limited to, reduction of the amount of contaminating DNA, ability to avoid preparation steps (e.g., extractions), speed, use of less enzyme, and the like. Exemplary procedures that may be improved by the compositions and methods of the present invention include those described by Kabir et al., J. Biosci. Bioeng. 96: 250, 2003; Chai et al., J. Clin. Lab Anal. 19: 182, 2005; Del Aguila et al., BMC Mol. Biol. 6: 9, 2005; Matthews et al., Biotechniques 32: 1412, 2002; and Koponen et al., Mol. Ther. 5: 220, 2002, each of which is herein incorporated by reference in their entireties.

The enzyme mixtures of the present invention may also be used to improve real-time RT-PCR methods that find use in research and clinical diagnostic methods, including, but not limited to, Taqman (Applied Biosystems, Foster City, Calif.; U.S. Pat. No. 5,210,015, herein incorporated by reference in its entirety); FullVelocity (Stratagene, La Jolla, Calif.; U.S. Pat. Nos. 6,548,250 and 6,528,254, herein incorporated by reference in their entireties); and GeneCode (Eragen Corporation, Madison, Wis.; U.S. Pat. Publ. No. 20020150900, herein incorporated by reference in its entirety).

In some embodiments, total destruction of DNA is desired. In some embodiments, the methods of the present invention eliminate all detectable DNA molecules in a sample. In some embodiments, the amount of DNA eliminated is greater than the amount eliminated using the type I DNase alone (e.g., as tested in side-by-side experiment using the same reactions conditions). In some embodiments of the present invention no contamination with genomic DNA from a cell lysate can be detected after 45 rounds of PCR amplification as measured by real-time PCR.

Enzyme mixtures of the present invention also find use in vivo for research and therapeutic applications. The enzyme mixtures may be used in place of type I DNase in any application where such type I DNase enzymes are used (see e.g., U.S. Pat. Nos. 6,440,412; 6,348,343; and 6,569,660; herein incorporated by reference in their entireties). For example, the enzyme mixtures of the present invention may be administered to a subject to reduce the viscosity of mucus. Patients that have pulmonary disease such as infectious pneumonia, bronchitis, tracheobronchitis, bronchiectasis, cystic fibrosis, asthma, TB, or fungal infections, atelectasis due to tracheal or bronchial impaction, and/or complications due to tracheostomy may be administered the enzyme mixtures of the present invention. Administration may be by any suitable means, including aerosolization of a solution of enzyme mixture.

In addition to direct uses, the enzyme mixtures may be used as an adjunctive treatment for the management of abscesses of closed space infections, emphysema, meningitis, peritonitis, sinusitis, otitis, periodontitis, pancreatitis, cholelithiasis, endocronditis, and septic arthritis. The enzyme mixture may also be used in topical or mucosal treatments of a variety of inflammatory and infected lesions, such as infected lesions of the skin and/or mucosal membranes, surgical wounds, ulcerative lesions, and burns.

The enzyme mixture may also be used for maintaining the flow in medical conduits communicating with a body cavity, including surgical drainage tubes, urinary catheters, peritoneal dialysis ports, intratracheal oxygen catheters, and junction ports for artificial organs that are in contact with a subject's vascular system.

In some preferred research and therapeutic embodiments, human DNase I is used with a complementary DNase (e.g., exonuclease I) to create an enzyme combination of the present invention. Human DNase I has been used to reduce the viscoelasticity of pulmonary secretions (mucus) in such diseases as pneumonia and cystic fibrosis (CF), thereby aiding in the clearing of respiratory airways. One such formulation is sold under the tradename PULMOZYME (dornase alfa, Genentech) in which recombinant human DNase I is provided to a patient in an inhaled solution that is sterile, clear, colorless, and contains a highly purified solution of DNase I. The DNase I was found to be effective in reducing the viscoelasticity of pulmonary secretions by hydrolyzing or degrading high-molecular-weight DNA that is present in the secretions. The present invention contemplates that a complementary DNase (e.g. exonuclease I) may be formulated with human type I DNase (e.g. human DNase I) and used in such methods to provide enhanced DNase function. In some embodiments, the complementary DNase is a human 3′ to 5′ exodeoxyribonuclease that digests single-stranded DNA hat has a 3′-hydoxyl group. In some embodiments, the complementary DNase is TREX1/DNase III (Hoss et al., EMBO J. 18: 3868, 1999; Morita et al., Mol Cell Biol, 24: 6719, 2004). The present invention also contemplates that the complementary DNase (e.g. exonuclease I) may be provided separate from but contemporaneous with PULMOZYME therapy in a manner that enhances the function of the DNase in the PULMOZYME product. It is contemplated that the combination of the present invention permits a treating physician to either use less DNase I (e.g., PULMOZYME), providing lower toxicity or lower immunogenic response, or to use the same amount of DNase I (e.g., PULMOZYME), but providing higher efficacy with the combination with the complementary DNase.

The present invention also provides kits configured for use, alone or in combination with other components, in any of the above methods.

### EXAMPLES

The following examples are provided in order to demonstrate and further illustrate certain preferred embodiments and aspects of the present invention, and are not to be construed as limiting the scope thereof.

### Background Information for Examples 1 and 2

1 Unit of DNase I activity is defined as the amount of enzyme that will degrade 1 μg of DNA in 10 min. at 37° C. The activity is determined in a buffer containing 10 mM Tris-HCl, pH 7.5; 2.5 mM MgCl₂, and 0.5 mM CaCl₂. The standard reaction volume is 50 μl. Dilutions of DNase I, when indicated, are made in DNase I storage buffer, which contains 50 mM Tris-HCl, pH 7.5; 10 mMCaCl₂; 10 mM MgCl₂; 0.1% Triton X-100, and 50% (v/v) glycerol. DNase I concentrations from various venders are typically 1 or 2 U/μl. However, vendors typically do end point assay on one of several DNA species (plasmid, phage lambda, etc.) and determine the unit concentration by visual inspection of DNA on a standard agarose gel. This is a subjective evaluation that results in differences between assessments of level of digestion.

Premixes for DNase I activity assays are as follows: 10× reaction buffer (100 mM Tris-HCl, pH 7.5; 25 mM MgCl₂, and 5 mM CaCl₂); DNA (1 μg/reaction); and water to a final volume of 49 μl per reaction. 49 μl of premix are dispensed into 0.5 ml Eppendorf tubes followed by 1 μl DNase I. After mixing, samples are incubated for 10 min. at 37° C. Reactions are stopped with 10 μl of a 6× Stop solution which contains 0.1 M EDTA, pH 7.5; 40% (w/v) sucrose, and 0.25% bromophenol blue followed by mixing and heating for 5 minutes at 70° C. Aliquots are subjected to electrophoresis on a 1% agarose gel run in Tris acetate EDTA buffer. Gels are stained with SYBRgold and are photographed with long UV wavelength transillumination.

### Example 1

This example demonstrated that residual DNA remains after DNase I digestion using commercially available DNase I enzymes. 1 μg amounts of the plasmid pUC 19 were incubated in DNase I buffer in a 50 μl reaction with 1 μl amounts of DNase I from various vendors. Reactions were incubated for 10 min. at 37° C. and subjected to agarose gel electrophoresis and staining as described above.

### Example 2

This example describes the testing of a variety of nucleases in an attempt to complete the digestion of DNA with Epicentre DNase I. pUC 19 (1 μg) was incubated in reaction buffer for 10 min. at 37° C. with the indicated nucleases. Results were analyzed by agarose gel electrophoresis as described earlier. Only exonuclease I was capable of eliminating residual DNA remaining after DNase I digestion under the conditions described.

### Example 3

This example describes the ability of enzyme mixtures of the present invention to enhance RT-PCR reactions by reducing contaminating genomic DNA.

HeLa cells were cultured with conventional method in a CO₂ incubator. The cells were grown in DMEM (Dulbecco's Modification of Eagle's Medium) with 4.5 g/L glucose, L-glutamine and sodium pyruvate, supplemented with 10% fetal bovine serum (Mediatech Inc., Herndon, Va.). 0.25% trypsin (Mediatech) was used to harvest the cells. Seven individual samples of approximately 8×10⁵ HeLa cells were harvested and washed with 1×PBS before subjected to RNA purification using MasterPure™ RNA Purification Kit (Epicentre Biotechnologies, Madison, Wis.). These seven RNA samples followed exactly the same purification procedure except that they were treated with 2 U of different versions of DNase I. One of the seven samples was not treated with any DNase. These samples were incubated for 10 minutes at 37° C. for the DNase treatment. Five of the samples were treated with DNase I alone from different vendors (Ambion's DNase I, Promega's RQ1 DNase I, Ambion's rDNase I, Ambion's Turbo DNase, and Epicentre's DNase I). The remaining sample was treated with DNase I (Epicentre) as well as exonuclease I (Epicentre). The RNA yield from each sample was measured using SpectraMax/M2 (Molecular Devices Corp., Sunnyvale, Calif.). The resulting RNA concentration was adjusted to 100 ng/μl for each sample.

The following components were included in each 25-μl qPCR reaction: 1×FailSafe™ PROBES Real-Time PCR Optimization PreMix P3 (Epicentre Biotechnologies, Madison, Wis.), 12.5 pmole of the forward and reverse PCR primers, 100 nM of 5′-Hex/3′-BHQ1 labeled sequence-specific probe, 5 μl or 500 ng of the above seven RNA samples, 1 U of FailSafe™ Real-Time PCR Enzyme Mix. The cycling conditions were 2 minutes at 95° C., followed by 45 cycles of 15″ at 94° C. and 90″ at 60° C. The PCR primers and probe were designed to detect a 426 bp fragment of β-actin gene.

When NTC (No Template Control (i.e., ddH₂O)) was added in place of PCR template there was no amplification based on qPCR quantification graphs, therefore indicating a clearly negative control. The RNA sample with no DNase treatment displayed a Ct of 20.5 cycles. Ambion's DNase I, Promega's RQ1 DNase I, Ambion's rDNase I, Ambion's Turbo DNase, and Epicentre's DNase I displayed a Ct of 37.6, 37.0, 35.9, 30.3, and 35.2 cycles respectively, resulted from minute amount of genomic DNA contamination. The sample treated with the enzyme mixture (DNase I and exonuclease I) displayed no amplification, hence no detectable genomic DNA. Therefore, the enzyme mixture demonstrated better elimination of HeLa genomic DNA contamination from the purified RNA sample than any of the commercially available products tested.

### Example 4

This example describes the ability enzyme mixtures of the present invention to enhance RT-PCR reactions by reducing contaminating genomic DNA.

HeLa cells were cultured with conventional method in a CO₂ incubator. The cells were grown in DMEM (Dulbecco's Modification of Eagle's Medium) with 4.5 g/L glucose, L-glutamine and sodium pyruvate, supplemented with 10% fetal bovine serum (Mediatech Inc., Hemdon, Va.). 0.25% trypsin (Mediatech) was used to harvest the cells. Seven individual samples of approximately 1.35×10⁷ HeLa cells were harvested and washed with 1×PBS before subjected to RNA purification using MasterPure™ RNA Purification Kit (Epicentre Biotechnologies, Madison, Wis.). These eight RNA samples followed exactly the same purification procedure except that they were treated with 1 U of different versions of DNase I. One of the eight samples was not treated with any DNase. These samples were incubated for 10 minutes at 37° C. for the DNase treatment. Six of the samples were treated with DNase I alone from different vendors (Ambion's DNase I, Promega's RQ1 DNase I, Ambion's rDNase I, Invitrogen's DNase I, Qiagen's DNase I, and Epicentre's DNase I). The remaining sample was treated with DNase I (Epicentre) as well as exonuclease I (Epicentre). The mixture of DNase I and exonuclease I is referred to as “DX.” The RNA yield from each sample was measured using SpectraMax/M2 (Molecular Devices Corp., Sunnyvale, Calif.). The resulting RNA concentration was adjusted to 1000 ng/μl for each sample.

The following components were included in each 25-μl qPCR reaction: 1×FailSafe™ PROBES Real-Time PCR Optimization PreMix P3 (Epicentre Biotechnologies, Madison, Wis.), 12.5 pmole of the forward and reverse PCR primers, 100 nM of 5′-Cy5/3′-BHQ2 labeled sequence-specific probe, 1000 ng of the above eight RNA samples, 1 U of FailSafe™ Real-Time PCR Enzyme Mix. The cycling conditions were 2 minutes at 95° C., followed by 45 cycles of 15″ at 94° C. and 90″ at 60° C. The PCR primers and probe were designed to detect a 317-bp fragment of human cyclophilin A gene.

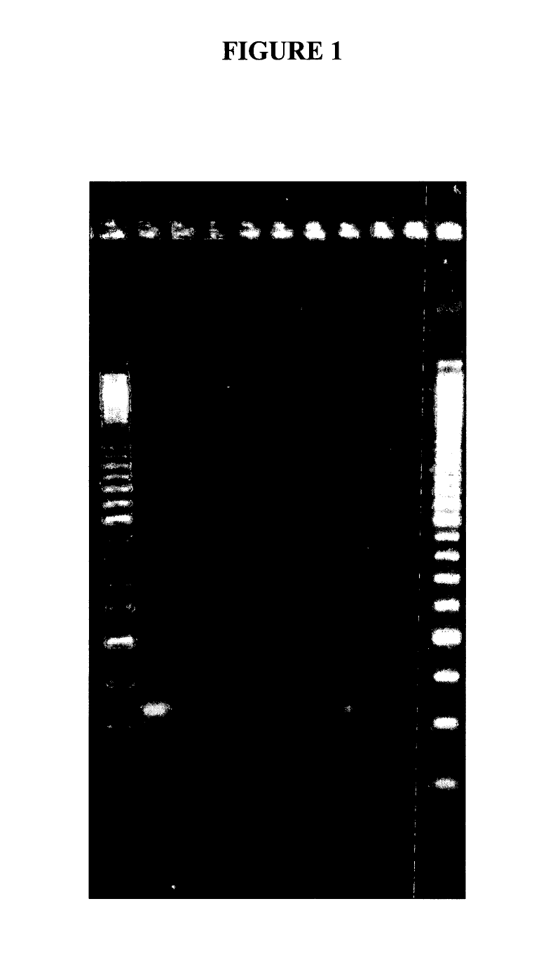

When NTC (No Template Control (i.e., ddH₂O)) was added in place of PCR template there was no amplification based on qPCR quantification graphs and gel electrophoresis analysis (FIG. 1), therefore indicating a clearly negative control. The RNA sample with no DNase treatment displayed a Ct of 19.4 cycles. Ambion's DNase I, Ambion's rDNase I, Promega's RQ1 DNase I, Invitrogen's DNase I, Qiagen's DNase I and Epicentre's DNase I displayed a Ct of 27.8, 28.2, 27.9, 27.6, 32.5, and 30.3 cycles respectively, resulted from minute amount of genomic DNA contamination. The sample treated with the DX enzyme mixture (DNase I and exonuclease I) displayed no amplification, hence no detectable genomic DNA. These PCR reactions were also analyzed by agarose gel electrophoresis and stained with SYBR Gold™ (Invitrogen, Calif.) (FIG. 1). Similar conclusions were drawn from the gel analysis. Therefore, the enzyme mixture of the present invention demonstrated better elimination of HeLa genomic DNA contamination from the purified RNA sample than any of the commercially available products tested. In FIG. 1, the following lane designations apply: 1: 100 bp ladder; 2: No DNase treatment; 3: DX-treated; 4: Epicentre's DNase I; 5: Ambion's DNase I; 6: Ambion's rDNase I; 7: Promega's RQ1 DNase I; 8: Invitrogen's DNase I; 9: Qiagen's DNase I; 10: No template PCR negative control (NTC); and 11: 100 bp ladder.

### Example 5

These same eight RNA samples were also used in standard PCR using primers designed to amplify a 426 bp fragment of human β-actin gene.

The following components were included in each 50-μl qPCR reaction: 1×FailSafe™ PCR PreMix C (Epicentre Biotechnologies, Madison, Wis.), 6.25 pmole of the forward and reverse PCR primers, 1000 ng of the above eight RNA samples, 2.5 U of FailSafe™ PCR Enzyme Mix. The cycling conditions were 2 minutes at 94° C., followed by 30 cycles of 15″ at 94° C., 30″ at 60° C., and 30″ at 72° C.

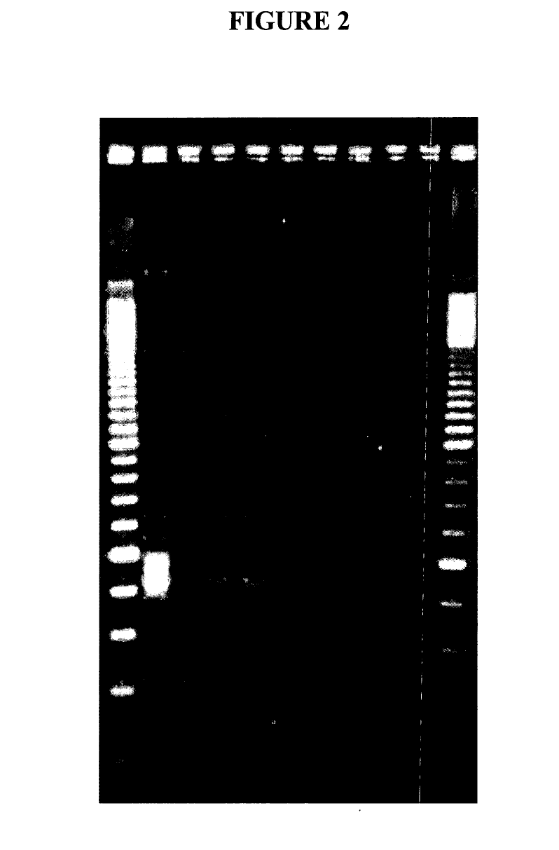

As demonstrated in FIG. 2, when NTC (No Template Control (i.e., ddH₂O)) was added in place of PCR template there was no amplification based on gel electrophoresis analysis, therefore indicating a clearly negative control. The RNA sample with no DNase treatment displayed strong PCR amplification as expected. Ambion's DNase I, Ambion's rDNase I, Promega's RQ1 DNase I, Invitrogen's DNase I, Qiagen's DNase I and Epicentre's DNase I all displayed a PCR product band corresponding to the expected 426 bp amplicon, resulted from minute amount of genomic DNA contamination. The sample treated with DX enzyme mixture (DNase I and exonuclease I) displayed no amplification, hence no detectable genomic DNA. Therefore, the enzyme mixture demonstrated better elimination of HeLa genomic DNA contamination from the purified RNA sample than any of the commercially available products tested. In FIG. 2, the following lane designations apply: 1: 100 bp ladder; 2: No DNase treatment; 3: DX-treated; 4: Epicentre's DNase I; 5: Ambion's DNase I; 6: Ambion's rDNase I; 7: Promega's RQ1 DNase I; 8: Invitrogen's DNase I; 9: Qiagen's DNase I; 10: No template PCR negative control (NTC); and 11: 100 bp ladder.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A composition comprising a purified type I DNase and a purified complementary DNase enzyme.

2. The composition of claim 1, wherein said type I DNase and said complementary DNase enzyme are provided together in a buffer.

3. The composition of claim 1, wherein said type I DNase and said complementary DNase enzyme are provided together in a reaction mixture.

4. The composition of claim 1, further comprising RNA molecules.

5. The composition of claim 1, further comprising cultured cells.

6. The composition of claim 1, further comprising a restriction enzyme.

7. The composition of claim 1, wherein said type I DNase is human, bovine, or porcine DNase I.

8. The composition of claim 1, wherein said type I DNase is recombinant type I DNase.

9. The composition of claim 1, wherein said complementary DNase enzyme is an exonuclease I enzyme.

10. A kit comprising a purified type I DNase and a purified complementary DNase enzyme.

11. The kit of claim 10, wherein said type I DNase and said complementary DNase enzyme are provided at at least 5-fold concentration excess of a reaction concentration.

12. The kit of claim 10, the kit further comprises a reaction buffer.

13. The kit of claim 10, wherein said type I DNase and said complementary DNase enzyme are provided together in the same vessel.

14. The kit of claim 10, further comprising an RNA molecule as a control.

15. The kit of claim 10, further comprising a DNA molecule as a control.

16. The kit of claim 10, further comprising a reverse transcriptase.

17. The kit of claim 10, further comprising a DNA polymerase.

18. The kit of claim 10, further comprising oligonucleotide primers.

19. The kit of claim 10, further comprising an RNA polymerase.

20. The kit of claim 10, further comprising an agent that inactivates type I DNase.

21. The kit of claim 10, wherein said type I DNase and said complementary DNase are contained in separate vessels.

22. The kit of claim 10, wherein said type I DNase is a human, bovine or porcine DNase I.

23. The kit of claim 10, wherein said type I DNase is recombinant type I DNase.

24. The kit of claim 10, wherein said complementary DNase enzyme is an exonuclease I enzyme.

25. A method of digesting DNA in a sample, comprising: treating a sample suspected of comprising DNA with an enzyme mixture comprising a purified type I DNase and a purified complementary DNase enzyme.

26. The method of claim 25, wherein said type I DNase and said complementary DNase enzyme are provided together in a buffer.

27. The method of claim 25, wherein said sample comprises RNA molecules.

28. The method of claim 27, further comprising the step of isolating said RNA molecules.

29. The method of claim 27, further comprising the step of treating the sample with a reverse transcriptase.

30. The method of claim 29, further comprising the step of treating the sample with a DNA polymerase.

31. The method of claim 27, further comprising the step of generating cDNA from said RNA.

32. The method of claim 31, further comprising the step of amplifying said cDNA using a polymerase chain reaction.

33. The method of claim 32, further comprising synthesizing RNA from said cDNA using an RNA amplification reaction.

34. The method of claim 25, wherein said sample comprises RNA molecules generated by an in vitro transcription reaction.

35. The method of claim 25, wherein said sample comprises cultured cells.

36. The method of claim 25, wherein said type I DNase is a DNase I enzyme.

37. The method of claim 25, wherein said complementary DNase enzyme is an exonuclease I enzyme.

38. A method for digesting DNA in mucus, comprising: exposing mucus suspected of comprising DNA to a purified type I DNase and a purified complementary DNase enzyme.

39. The method of claim 38, wherein said type I DNase is a DNase I enzyme.

40. The method of claim 38, wherein said complementary DNase enzyme is an exonuclease I enzyme.
